(19) United States
(12) Patent Application Publication
(10) Pub. No.: US 2022/0313962 A1
(43) Pub. Date: Oct. 6, 2022

(54) GUIDEWIRE CONTROLLER CASSETTE AND USING METHOD THEREOF

(71) Applicants: XCATH, INC., Houston, TX (US); THE BOARD OF REGENTS OF THE UNIVERSITY OF TEXAS SYSTEM, Austin, TX (US)

(72) Inventors: Daniel H. KIM, Houston, TX (US); Dong Suk SHIN, Houston, TX (US)

(21) Appl. No.: 17/709,155

(22) Filed: Mar. 30, 2022

Related U.S. Application Data
(60) Provisional application No. 63/169,637, filed on Apr. 1, 2021.

(51) Int. Cl. A61M 25/09

(52) U.S. Cl. CPC A61M 25/09041; A61M 2205/103

(57) ABSTRACT

Provided herein as a guidewire controller cassette for positioning a guidewire within a patient body, including a housing having an interior space; a translational module received within the interior space and having an entry side, an opposed exit side and a lateral wall being disposed therebetween, the translational module comprising a first guidewire path extending between the entry side and the exit side and configured to move the guidewire translationally along the first guidewire path; and a rotational module received within the interior space and mounted at the lateral wall, the rotational module comprising an opening for receiving the proximal end of the guidewire, a rotating axis allowing the proximal end of the guidewire being rotated thereabout, and a second guidewire path which is a loop path extending out from the opening to the entry side.

### CROSS REFERENCE TO RELATED APPLICATION

This application claims benefit of U.S. provisional patent application Ser. No. 63/169,637, filed Apr. 1, 2021, which is herein incorporated by reference.

### BACKGROUND

### Field

The present specification relates to medical devices and controlling methods used for minimally invasive interventional procedures, more particularly to the field of robotic controller for endovascular interventions using guidewires and catheters to employ a distal tip to a targeted site thereof within human lumens.

### Description of the Related Art

Guidewires are used to guide a secondary sheath (e.g. a catheter), which is fed along and over the guidewire, to a desired location in a body, for example a mammalian body such as a human body. In one application for minimally invasive interventional procedures, a guidewire is introduced into a body lumen, i.e., a blood vessel, through an incision through the patient's skin and the lumen wall, and the introduced, or distal, end of the guidewire is guided therefrom to a desired location of the lumen, or of a lumen which branches into, or from, the lumen into which the guidewire is introduced.

One issue with guidewire introduction systems is the limited ability to conform the distal end of the guidewire to follow tortuous lumen geometries, as well as to guide the distal end into an intersecting lumen or branch lumen connected to the lumen within which the distal end of the guidewire is positioned. To guide the distal end of the guidewire into a branch lumen, the distal end of the guidewire must be controllably moved from alignment with the lumen in which it reached the branching lumen location to an alignment whereby further movement of the guidewire inwardly of the body will cause the guidewire to enter and follow the branch lumen. In some cases, the branch lumen, a location of which is the target destination of the distal end of the guidewire, intersects the lumen in which the distal end is present at a large angle, for example greater than forty-five, degrees, and in some cases greater than ninety degrees. In other cases, it could be difficult to guide the distal end of the guidewire into the tortuous and sharp turns of vessels without damaging the lumen because of the tendency of the turns and fluid flows adjacent to sidewalls that would push the guidewire towards the sidewall of the vessels.

To overcome such issue, one methodology for facilitating the control of the orientation of the distal end of the guidewire includes developing robotic systems for stable control of the movement of catheters and guidewires surrounding thereover. For example, US U.S. Ser. No. 10/342,953B2 discloses a robotic catheter system. The catheter system includes a housing and a drive mechanism supported by the housing. The drive mechanism includes an engagement structure configured to engage and to impart movement to a catheter device. A cassette for use with the robotic catheter system is also provided. The cassette includes a housing, a first axial drive mechanism supported by the housing to releasably engage and drive a guide wire along a longitudinal axis of the guide wire, a second axial drive mechanism supported by the housing to releasably engage and drive a working catheter along a longitudinal axis of the working catheter, and a rotational drive mechanism supported by the housing to rotate the guide wire about its longitudinal axis.

### SUMMARY

To overcome the above-mentioned challenges, there is still an urgent need for a novel robotic control system for easy navigation and excellent stability and have the potential to reduce radiation exposure to patients and surgeons while achieving more uniform operator-independent outcome.

Here, in one aspect, a guidewire controller cassette is provided for moving a guidewire having a proximal end. The guidewire controller comprises: a housing having an interior space; a translational module received within the interior space and having an entry side, an opposed exit side and a lateral wall being disposed therebetween, the translational module comprising a first guidewire path extending between the entry side and the exit side and configured to move the guidewire translationally along the first guidewire path; and a rotational module received within the interior space and mounted at the lateral wall, the rotational module comprising an opening for receiving the proximal end of the guidewire, a rotating axis allowing the proximal end of the guidewire being rotated thereabout, and a second guidewire path which is a loop path extending out from the opening to the entry side.

In another aspect, a method for moving a guidewire is provided. The method comprises: providing the guidewire controller comprising: a cartridge having an interior space; a translational module received within the interior space and having an entry side, an opposed exit side and a lateral wall being disposed therebetween, the translational module comprising a first guidewire path extending between the entry side and the exit side and configured to move the guidewire translationally along the first guidewire path; and a rotational module received within the interior space and mounted at the lateral wall, the rotational module comprising an opening for receiving the proximal end of the guidewire and a longitudinal axis allowing the proximal end of the guidewire being rotated thereabout, and a second guidewire path which is a loop path extending out from the opening to the entry side; engaging the guidewire to the rotational module and the translational module along the first guidewire path and the second guidewire path; and moving the guidewire translationally or rotationally along the first guidewire path and the second guidewire path in response to a control signal of the guidewire controller.

### BRIEF DESCRIPTION OF THE DRAWINGS

So that the manner in which the above-recited features can be understood in detail, reference is made to the following detailed description taken in conjunction with the accompanying drawings, in which:

FIG. 1 is a perspective view of a simplified multiple axial catheter system according to some examples.

FIG. 2 is a perspective view of a multiple axial catheter system according to some examples.

FIGS. 3A, 3B, 3C, and 3D are a perspective view, a side view, a top view, and a bottom view of a proximal drive unit according to some examples.

FIGS. 4A, 4B, 4C, and 4D are a perspective view, a side view, a top view, and a bottom view of an intermediate drive unit according to some examples.

FIGS. 5A, 5B, 5C, and 5D are a perspective view, a side view, a top view, and a bottom view of a distal drive unit according to some examples.

FIG. 6 is an exploded perspective view of a drive assembly according to some examples.

FIG. 7A is a perspective view of a proximal cassette according to some examples.

FIGS. 7B and 7C are respective perspective views of the proximal cassette of FIG. 7A partially disassembled according to some examples.

FIGS. 7D, 7E, 7F, and 7G are back, front, top, and bottom views, respectively, of the partially disassembled proximal cassette of FIG. 7C according to some examples.

FIGS. 8A and 8B are schematic illustrations depicting rotation and advancement, respectively, of a guidewire according to some examples.

FIGS. 9A and 9B and 9C are exploded perspective views of a translation assembly mechanically coupled to a track according to some examples, and a schematic view of the proximal cassette having the translation assembly located therein according to some examples.

FIG. 10 is an exploded perspective view of a clasp assembly according to some examples.

FIGS. 11A and 11B are exploded perspective views of respective portions of a pinch and advance assembly according to some examples.

FIG. 12 is an exploded perspective view of a follower assembly according to some examples.

FIG. 13 is an exploded perspective view of a catheter rotation assembly according to some examples.

FIG. 14 is an exploded perspective view of a guidewire rotation assembly according to some examples.

FIGS. 15, 16, and 17 are configurations that include a catheter and a Y-connector according to some examples.

FIGS. 18 and 19 are schematic illustrations depicting rotation and advancement, respectively, of a catheter according to some examples.

### BRIEF DESCRIPTION

Examples described herein generally relate to a system and a method for endovascular procedures. More specifically, some examples described herein enable a robotic system, and methods of operating such a robotic system, for endovascular procedures. In some examples, a multiple axial catheter system can include multiple drive units each for a respective cassette. The multiple drive units can collectively enable advancement and rotation of a number of catheters and a guidewire. Advancement and rotation of a catheter or guidewire in such a system can be independent of any advancement or rotation of any other catheter or guidewire. Some examples include a cassette that can implement advancement and/or rotation of a catheter or guidewire.

Examples described herein can achieve various benefits. A robotic system can permit a surgeon performing an endovascular procedure precise control of the navigation of an endovascular insertion device (e.g., a catheter and/or a guidewire). Rotation of an endovascular insertion device in an endovascular procedure can permit the endovascular insertion device to be guided through tortuous vasculature in a body undergoing the endovascular procedure. Further, a rotation assembly that is configured to rotate an endovascular insertion device can remain mechanically coupled to the endovascular insertion device (e.g., including while the endovascular insertion device is advanced), which can maintain a rotational orientation of the endovascular insertion device.

Various features are described hereinafter with reference to the figures. An illustrated example need not have all the aspects or advantages shown. An aspect or an advantage described in conjunction with a particular example is not necessarily limited to that example and can be practiced in any other examples even if not so illustrated or if not so explicitly described. Further, methods described herein may be described in a particular order of operations, but other methods according to other examples may be implemented in various other orders (e.g., including different serial or parallel performance of various operations) with more or fewer operations. Various figures are illustrated with three-dimensional coordinate axes for orienting figures with respect to each other, although such axes may not be explicitly described below. The three-dimensional coordinate axes show a directionality of a positive direction of movement along a respective axis. More specifically, the three-dimensional coordinate axes show a positive X (+X) direction, a positive Y (+Y) direction, and a positive Z (+Z) direction.

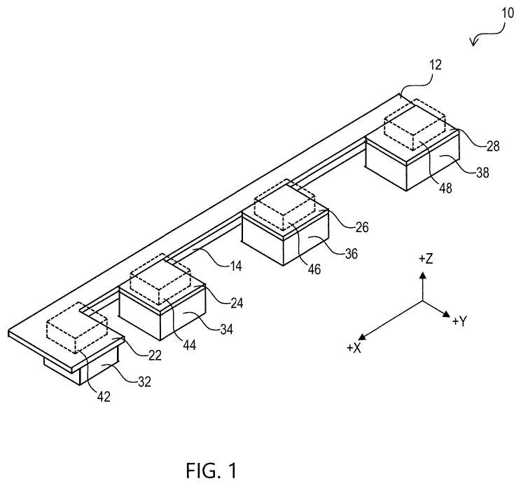

FIG. 1 illustrates a perspective view of a simplified multiple axial catheter system 10 according to some examples. The multiple axial catheter system 10 includes a frame 12, a track 14, cassette platforms 22, 24, 26, 28, and drive units 32, 34, 36, 38. In operation, the multiple axial catheter system 10 implements a distal cassette 42, a first intermediate cassette 44, a second intermediate cassette 46, and a proximal cassette 48. The cassettes 42-48 may be single use cassettes (e.g., usable for a single endovascular procedure). The illustrated multiple axial catheter system 10 implements cassette platforms and drive units for two intermediate cassettes. In other examples, cassette platforms and drive units for fewer or more (e.g., one, three, four, etc.) intermediate cassettes can be implemented.

The frame 12 is a support structure to which the various other components of the multiple axial catheter system 10 are mechanically coupled and supported. Although not illustrated, a casing or shroud can be included with and around the frame 12. The track 14 underlies and is mechanically coupled to the frame 12. The track 14 extends along a longitudinal axis of the frame 12, which is in an X-direction in FIG. 1. The track 14 permits translation of components mechanically coupled to and moveable along the track 14 in a direction parallel to the longitudinal axis (e.g., in an X-direction).

The distal cassette platform 22 is illustrated as being integral to the frame 12, and in other examples, the distal cassette platform 22 can be otherwise mechanically coupled to the frame 12, such as by brackets and/or other framing. The distal drive unit 32 underlies the distal cassette platform 22 and is mechanically coupled to the distal cassette platform 22 and/or the frame 12. The distal cassette platform 22 and the distal drive unit 32 are at a fixed position relative to the frame 12 (e.g., by the mechanical coupling to the frame 12). In operation, the distal cassette 42 is disposed on the distal cassette platform 22, and can be mechanically attached to the distal cassette platform 22 and/or the distal drive unit 32.

The first intermediate cassette platform 24 and/or the first intermediate drive unit 34 are mechanically coupled to and moveable along the track 14. The first intermediate drive unit 34 underlies and is mechanically coupled to the first intermediate cassette platform 24. The first intermediate cassette platform 24 and the first intermediate drive unit 34 are configured to and capable of being translated along a direction parallel to the longitudinal direction of the track 14 (e.g., in an X-direction). In operation, the first intermediate cassette 44 is disposed on the first intermediate cassette platform 24, and can be mechanically attached to the first intermediate cassette platform 24 and/or the first intermediate drive unit 34.

The second intermediate cassette platform 26 and/or the second intermediate drive unit 36 are mechanically coupled to and moveable along the track 14. The second intermediate drive unit 36 underlies and is mechanically coupled to the second intermediate cassette platform 26. The second intermediate cassette platform 26 and the second intermediate drive unit 36 are configured to and capable of being translated along a direction parallel to the longitudinal direction of the track 14 (e.g., in an X-direction). In operation, the second intermediate cassette 46 is disposed on the second intermediate cassette platform 26, and can be mechanically attached to the second intermediate cassette platform 26 and/or the second intermediate drive unit 36.

The proximal cassette platform 28 and/or the proximal drive unit 38 are mechanically coupled to and moveable along the track 14. The proximal drive unit 38 underlies and is mechanically coupled to the proximal cassette platform 28. The proximal cassette platform 28 and the proximal drive unit 38 are configured to and capable of being translated along a direction parallel to the longitudinal direction of the track 14 (e.g., in an X-direction). In operation, the proximal cassette 48 is disposed on the proximal cassette platform 28, and can be mechanically attached to the proximal cassette platform 28 and/or the proximal drive unit 38. Hereinafter, the proximal cassette platform 28 and its components will be discussed in detail.

As illustrated, the first intermediate cassette platform 24 (with corresponding first intermediate drive unit 34) is disposed between and is moveable along the track 14 between the distal cassette platform 22 (with corresponding distal drive unit 32) and the second intermediate cassette platform 26 (with corresponding second intermediate drive unit 36). Similarly, the second intermediate cassette platform 26 (with corresponding second intermediate drive unit 36) is disposed between and is moveable along the track 14 between the first intermediate cassette platform 24 (with corresponding first intermediate drive unit 34) and the proximal cassette platform 28 (with corresponding proximal drive unit 38). Further, the proximal cassette platform 28 (with corresponding proximal drive unit 38) is disposed at a proximal position relative to the second intermediate cassette platform 26 (with corresponding second intermediate drive unit 36) and is moveable along the track 14 within such relative proximal position.

In operation, each cassette 42, 44, 46, in conjunction with the respective drive unit 32, 34, 36, is configured to advance a respective catheter (e.g., feed the respective catheter into a body or retrieve the catheter from the body). A given catheter that is advanced by a given cassette 42, 44, 46 has a proximal end mechanically coupled to a Y-connector secured by the next more proximally positioned cassette 44, 46, 48. For example, a catheter that is advanced by the distal cassette 42 has a proximal end mechanically coupled to a Y-connector secured by the first intermediate cassette 44; a catheter that is advanced by the first intermediate cassette 44 has a proximal end mechanically coupled to a Y-connector secured by the second intermediate cassette 46; and a catheter that is advanced by the second intermediate cassette 46 has a proximal end mechanically coupled to a Y-connector secured by the proximal cassette 48. Hence, advancing a catheter by a cassette can result in translation of the next more proximally positioned cassette and corresponding cassette platform and drive unit. The multiple axial catheter system 10 can further include one or more translation assemblies that can cooperatively translate a cassette (and corresponding cassette platform and drive unit) when a catheter that has a proximal end that the cassette secures is advanced, which can reduce or prevent tension on the catheter and buckling of the catheter. Additionally, the proximal cassette 48 is configured to advance a guidewire.

Additionally, each cassette 44, 46, 48 is configured to rotate a respective catheter that has a proximal end mechanically coupled to a Y-connector secured by that cassette 44, 46, 48. Further, the proximal cassette 48 is configured to rotate the guidewire.

In the multiple axial catheter system 10 of FIG. 1, each catheter and guidewire can be advanced independently of each other catheter and guidewire. Additionally, each catheter and guidewire can be rotated independently of each other catheter and guidewire. Details of such operations and details of components to implement such operations are described below in the context of various examples.

Additionally, each cassette 44, 46, 48 is configured to rotate a respective catheter that has a proximal end mechanically coupled to a Y-connector secured by that cassette 44, 46, 48. Further, the proximal cassette 48 is configured to rotate the guidewire.

In the multiple axial catheter system 10 of FIG. 1, each catheter and guidewire can be advanced independently of each other catheter and guidewire. Additionally, each catheter and guidewire can be rotated independently of each other catheter and guidewire. Details of such operations and details of components to implement such operations are described below in the context of various examples.

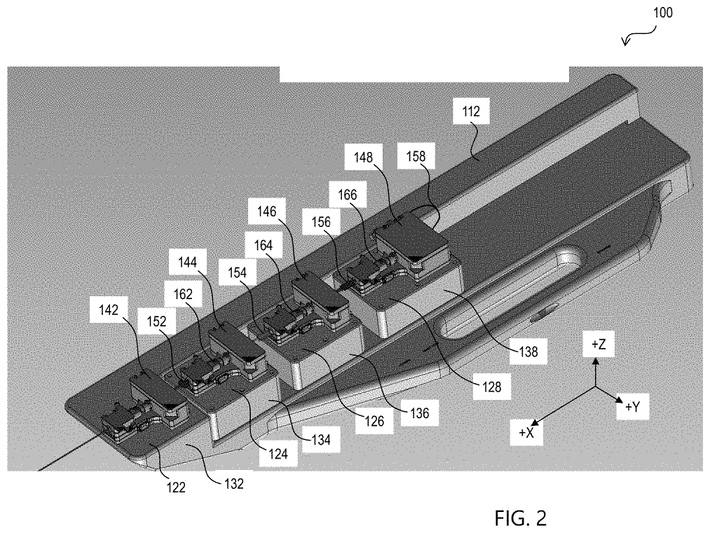

FIG. 2 illustrates a perspective view of a multiple axial catheter system 100 according to some examples. The multiple axial catheter system 100 of FIG. 2 illustrates more details of the simplified multiple axial catheter system 10 of FIG. 1. The multiple axial catheter system 100 similarly includes a frame 112, a track (occluded), cassette platforms 122, 124, 126, 128, and drive units 132, 134, 136, 138 (e.g., contained within a respective housing attached to the respective cassette platform 122-128). Here, distal drive unit 132 corresponds to the distal drive unit 32 of FIG. 1, the first intermediate drive unit 134 corresponds to the first intermediate drive unit 34 of FIG. 1, the second intermediate drive unit 136 corresponds to the second intermediate drive unit 36 of FIG. 1, and the proximal drive unit 48 corresponds to the proximal drive unit 48 of FIG. 1. FIG. 2 also shows cassettes 142, 144, 146, 148 mechanically attached to respective cassette platforms 122, 124, 126, 128 and/or drive units 132, 134, 136, 138. The cassettes 142-148 may be single use cassettes (e.g., usable for a single endovascular procedure). A person of ordinary skill in the art will readily understand the correspondence of these components of FIG. 2 with those shown in FIG. 1 and described above. FIG. 2 illustrates the cassettes 144-148 translated to distal positions along the track.

FIG. 2 further shows catheters 152, 154, 156, guidewire 158, and Y-connectors 162, 164, 166. The second catheter 154, driven by and supported on the second drive unit 134, is advanced through a bore of the first catheter 152 driven by and supported on the first drive unit 132. The third catheter 156, driven by and supported on the third drive unit 136 is advanced through a bore of the second catheter 154 driven by and supported on the second drive unit 135. The guidewire 158, driven and supported on the fourth drive unit 138 is advanced through a bore of the third catheter 156 driven by and supported on the third drive unit 136. Here, an inner diameter of the first catheter 152 (e.g., diameter of the bore) is greater than an outer diameter of the second catheter 154; an inner diameter of the second catheter 154 (e.g., diameter of the bore) is greater than an outer diameter of the third catheter 156; and inner diameter of the third catheter 156 (e.g., diameter of the bore) is greater than an outer diameter of the guidewire 158. In some instances, the first catheter 152 may be referred to as a guide catheter; the second catheter 154 may be referred to as an intermediate catheter; and the third catheter 156 may be referred to as a microcatheter.

The first catheter 152 has a female Luer lock connector at a proximal end thereof, which is mechanically coupled to a male Luer lock connector of the Y-connector 162. The Y-connector 162 is secured by the first intermediate cassette 144. The second catheter 154 has a female Luer lock connector at a proximal end, which is mechanically coupled to a male Luer lock connector of the Y-connector 164. The Y-connector 164 is secured by the second intermediate cassette 146. The third catheter 156 has a female Luer lock connector at a proximal end, which is mechanically coupled to a male Luer lock connector of the Y-connector 166. The Y-connector 166 is secured by the proximal cassette 148. A female Luer lock connector of a catheter can be mechanically coupled to a male Luer lock connector of a Y-connector by direct connection or by an intervening component. Some examples are described subsequently.

The distal cassette 142 (in conjunction with the distal drive unit 132) is configured to advance the first catheter 152, and the first intermediate cassette 144 (in conjunction with the first intermediate drive unit 134) is configured to rotate the first catheter 152. The first intermediate cassette 144 (in conjunction with the first intermediate drive unit 134) is configured to advance the second catheter 154, and the second intermediate cassette 146 (in conjunction with the second intermediate drive unit 136) is configured to rotate the second catheter 154. The second intermediate cassette 146 (in conjunction with the second intermediate drive unit 136) is configured to advance the third catheter 156, and the proximal cassette 148 (in conjunction with the proximal drive unit 138) is configured to rotate the third catheter 156. The proximal cassette 148 (in conjunction with the proximal drive unit 138) is configured to advance and rotate the guidewire 158.

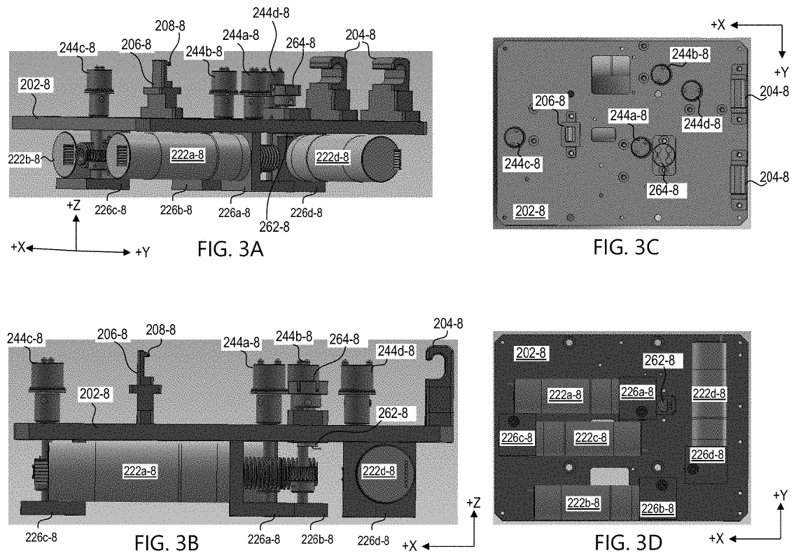

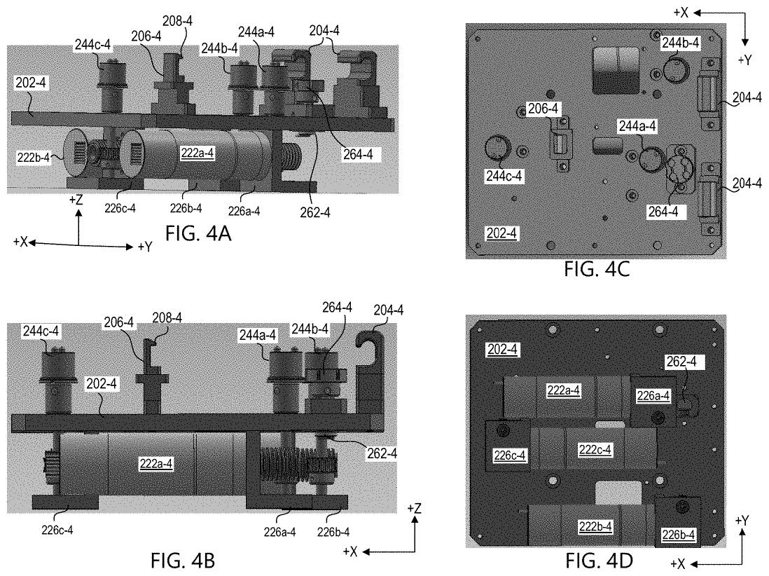

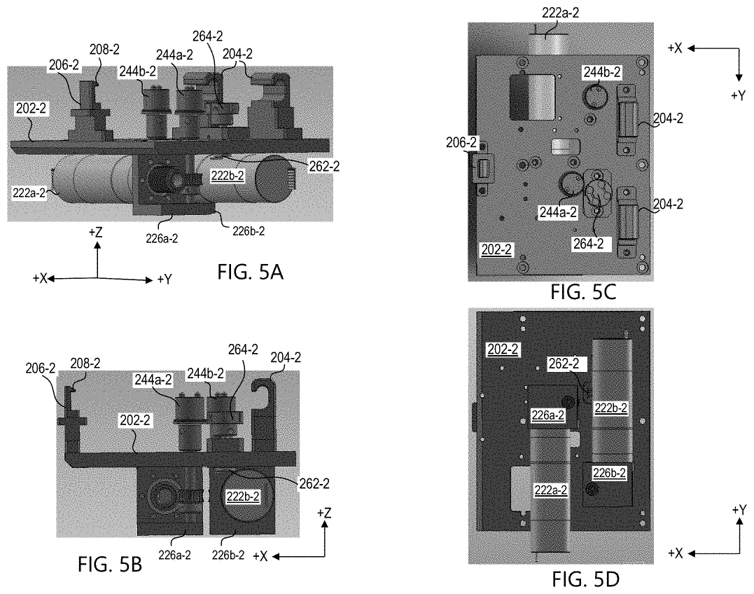

FIGS. 3A, 3B, 3C, and 3D are a perspective view, a side view, a top view, and a bottom view of the proximal drive unit 138 according to some examples. FIGS. 4A, 4B, 4C, and 4D are a perspective view, a side view, a top view, and a bottom view of an intermediate drive unit (e.g., the first intermediate drive unit 134 and the second intermediate drive unit 136) according to some examples. FIGS. 5A, 5B, 5C, and 5D are a perspective view, a side view, a top view, and a bottom view of the distal drive unit 132 according to some examples. The drive units shown in FIGS. 3A-3D through 5A-5D include some common components. To avoid redundant description, components in the figures are appended with a “−8”, “−4”, or “−2” to indicate in which of the proximal drive unit 138, intermediate drive units 134, 136, or distal drive unit 132, respectively, in which a given component is included. However, description of such components may be without reference to the appended “−8”, “−4”, or “−2”. Various modifications to such components between the different drive units, including different orientations, may be apparent to a person having ordinary skill, such as to accommodate different components, to accommodate different sized catheters or guidewire, etc.

Each drive unit of the proximal drive unit 138, intermediate drive units 134, 136, and distal drive unit 132 includes a support plate 202. The support plate 202 mechanically supports and is mechanically coupled to the components of the respective drive unit. The support plates 202 can vary in size, or layout or both as between different drive units to accommodate different components and/or different sizes of components.

Each drive unit includes a pair of hooks 204 and a clasp rib 206 mounted on the side of the support plate 202 that will be proximate the respective cassette platform during operation. The hooks 204 each have an interior surface that is a partial cylinder, where that cylinder is defined by a radius extending from a longitudinal center of the cylinder. The hooks 204 are mounted so that the longitudinal centers of the partial cylinders that define the interior surfaces of the hooks 204 are aligned, e.g., along a y-direction (as shown in the “B” side views). The clasp rib 206 is mounted on the support plate 202 such that a rib 208 of the clasp rib 206 is facing and opposing the openings of the hooks 204. The rib 208 has an upper inclined planar surface and a lower planar surface. As will become more apparent subsequently, the hooks 204 and clasp rib 206 are configured to secure a cassette. When installing a cassette, respective tabs of the cassette engage the hooks 204, followed by a spring-loaded clasp of the cassette engaging the clasp rib 206. The spring-loaded clasp has an inversely inclined planar surface that first contacts the upper inclined planar surface of the rib 208, which causes the clasp of the cassette to be displaced. Once the clasp passes the rib 208, the spring returns the clasp to be secured against the rib 208, thereby securing the cassette.

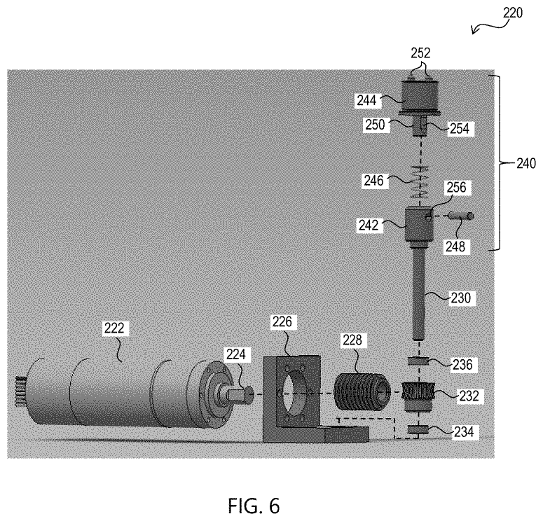

Each drive unit includes multiple drive assemblies. FIG. 6 illustrates an exploded perspective view of a drive assembly 220 according to some examples. The drive assembly 220 of FIG. 6 is used as the drive assemblies in the drive units, although different drive assemblies and/or modifications to the illustrated drive assembly 220 may be implemented in the drive units. Various types of shafts and gears are described below with respect to the drive assembly 220; however, other types of shafts and gears can be implemented to achieve a different configuration or orientation of a drive assembly.

The drive assembly 220 includes a rotational actuator 222. The rotational actuator 222 includes a drive shaft 224 (e.g., a shaft having a D-shaped cross-section normal to an axis of rotation of the shaft). The rotational actuator 222 is configured to rotate the drive shaft 224. In some examples, the rotational actuator 222 is a motor, such as an electric motor. In some instances, the rotational actuator 222 can be a servomotor. The rotational actuator 222 is mechanically attached to and mounted on a bracket 226, which is mechanically attached to and mounted on a support plate 202 (as shown in other figures). A worm gear 228 is mechanically attached to the drive shaft 224.

The drive assembly 220 further includes a transverse shaft 230 (e.g., a shaft having a D-shaped cross-section normal to an axis of rotation of the shaft). A gear 232 (e.g., a spur gear with outward faces that are concave) is mechanically attached to and encircles the transverse shaft 230. A ball bearing 234 mechanically couples the transverse shaft 230 to the bracket 226. A ball bearing 236 mechanically couples the transverse shaft 230 to and through an opening of the support plate 202. The ball bearings 234, 236 are disposed on the transverse shaft 230 on opposing sides of the gear 232.

The worm gear 228 engages the gear 232. The rotational actuator 222 is configured to rotate the drive shaft 224, which causes the worm gear 228 to rotate around a drive axis. Rotation of the worm gear 228 causes rotation of the gear 232 around a transverse axis that is transverse to the drive axis. Rotation of the gear 232 causes the transverse shaft 230 to rotate around the transverse axis.

The drive assembly 220 also includes a coupling assembly 240. The coupling assembly 240 includes a hollow, open-ended cylinder 242, a male connector 244, a spring 246, and a pin 248. The cylinder 242 (e.g., a closed end of the cylinder 242) is disposed on the end of the transverse shaft 230 opposite from where the transverse shaft 230 is mechanically coupled (by ball bearing 234) to the bracket 226. A longitudinal center axis of the cylinder 242 is co-linear with the transverse axis. The male connector 244 includes a solid cylinder. The solid cylinder has a piston 250 extending from a (bottom) circular surface and has coupling projections 252 extending from another, opposing (top) circular surface. One or more of the projections 252 extends in a direction parallel to the transverse axis at a position that is off of or displaced from the transverse axis. Thus, rotation of the piston 250 causes movement on the projections in a circular path generally centered on the longitudinal axis of the piston 250. The spring 246 and the piston 250 are disposed within the hollow region of the cylinder 242. The piston 250 has an elongated opening 254 through the piston 250 in a direction perpendicular to the transverse axis, and the elongated opening 254 is elongated in a direction along the transverse axis. The cylinder 242 has an opening 256 through a sidewall. With the spring 246 and piston 250 situated in the cylinder 242, the pin 248 is inserted, along a direction perpendicular with the transverse axis, through the opening 256 of the cylinder 242 and the elongated opening 254 of the piston 250. The pin 248 thereby secures the spring 246 and the piston 250 in the cylinder 242.

The elongated opening 254, by being elongated in a direction along the transverse axis, permits the piston 250, and thereby the male connector 244, to be translated along the transverse axis, i.e., move in the direction of the transverse axis. The pin 248 being inserted through the elongated opening 254 restricts such translation movement to the extent permitted by the elongation of the elongated opening 254. In the absence of any other force, the spring 246 exerts a force on the piston 250 in a direction away from the transverse shaft 230 causing the male connector 244 to be extended to the extent permitted from the transverse shaft 230. The permitted translation of the male connector 244 permits various tolerances to be accommodated when coupling the male connector 244 with a female connector of a cassette.

When the transverse shaft 230 rotates around the transverse axis, the cylinder 242 likewise rotates due to the mechanical connection between the transverse shaft 230 and the cylinder 242. The pin 248 being inserted in a direction perpendicular to the transverse axis cases the rotation of the cylinder 242 to be carried to the piston 250, and thereby, the male connector 244. The off-axis positioning of the projections 252 causes the rotation of the male connector 244 to be carried to a female connector of a cassette when mechanically coupled together.

Referring back to FIGS. 3A-3D through 5A-5D, each drive unit of the proximal drive unit 138, intermediate drive units 134, 136, and distal drive unit 132 includes an advance drive assembly 220a and a pinch drive assembly 220b. Referring to FIGS. 3A-3D and 4A-4D, the proximal drive unit 138 and intermediate drive unit 134, 136 each include a catheter rotational drive assembly 220c. Referring to FIGS. 3A-3D, the proximal drive unit 138 includes a guidewire rotational drive assembly 220d. Although the drive assemblies 220a, 220b, 220c, 220d are not explicitly identified in FIGS. 3A-3D through 5A-5D, some components of the drive assemblies 220a, 220b, 220c, 220d are identified and have a “a”, “b”, “c”, or “d” appended to the corresponding reference number from FIG. 6. The appended “a”, “b”, “c”, or “d” corresponds to drive assemblies 220a, 220b, 220c, 220d, respectively. As shown, for each of the drive assemblies 220a, 220b, 220c, 220d, the respective bracket 226 with the rotational actuator 222 mechanically attached thereto is mechanically attached to and mounted on a bottom side of the respective support plate 202. The respective transverse shaft 230 extends through an opening through the support plate 202, and the male connectors 244 extends away from a top side of the support plate 202.

Each drive unit of the proximal drive unit 138, intermediate drive unit 134, 136, and distal drive unit 132 includes an encoder 262 and encoder coupler 264. The encoder 262 is mounted through an opening through the support plate 202 and includes a shaft. The encoder coupler 264 is mechanically attached to the shaft of the encoder 262. The encoder coupler 264 extends away from the top side of the support plate 202. The encoder 262 is configured to detect a rotational position of the shaft of the encoder 262, which is rotated through the encoder coupler 264. As will be described in more detail subsequently, the encoder 262 detects a rotational position of the shaft such that a controller can determine a length and direction that an endovascular insertion device (e.g., a catheter or guidewire) has been advanced by the respective cassette. The encoder 262 can be used as a feedback for controlling advancement of the endovascular insertion device.

Each drive unit can also include other components not illustrated in the figures. For example, each drive unit can include electrical components (e.g., a controller, a circuit board, wires, and connectors) to enable operation of the rotational actuators 222. Each drive unit can include a sensor to sense when a cassette has been secured to the drive unit so that, e.g., a controller can prevent operation of any rotational actuator 222 while the sensor senses that no cassette has been secured to the drive unit. A spring-loaded release can be included to apply a force to any secured cassette, and the spring-loaded release can assist in de-coupling the cassette while the cassette is being removed from the drive unit.

In the context of the multiple axial catheter system 100 shown in FIG. 2, the top side of the support plate 202 of a drive unit is mechanically attached to a bottom side of a respective cassette platform 122, 124, 126, 128. The top side of the support plate 202-2 of the distal drive unit 132 is mechanically attached to a bottom side of the distal cassette platform 122. The top side of the support plate 202 (e.g., support plate 202-4) of the first intermediate drive unit 134 is mechanically attached to a bottom side of the first intermediate cassette platform 124. The top side of the support plate 202 (e.g., support plate 202-4) of the second intermediate drive unit 136 is mechanically attached to a bottom side of the second intermediate cassette platform 126. The top side of the support plate 202-8 of the proximal drive unit 138 is mechanically attached to a bottom side of the proximal cassette platform 128. For each drive unit and respective cassette platform, the hooks 204, the clasp rib 206, the male connectors 244 of the drive assemblies 220, and the encoder coupler 264 of the drive unit extend through the cassette platform for coupling with a cassette.

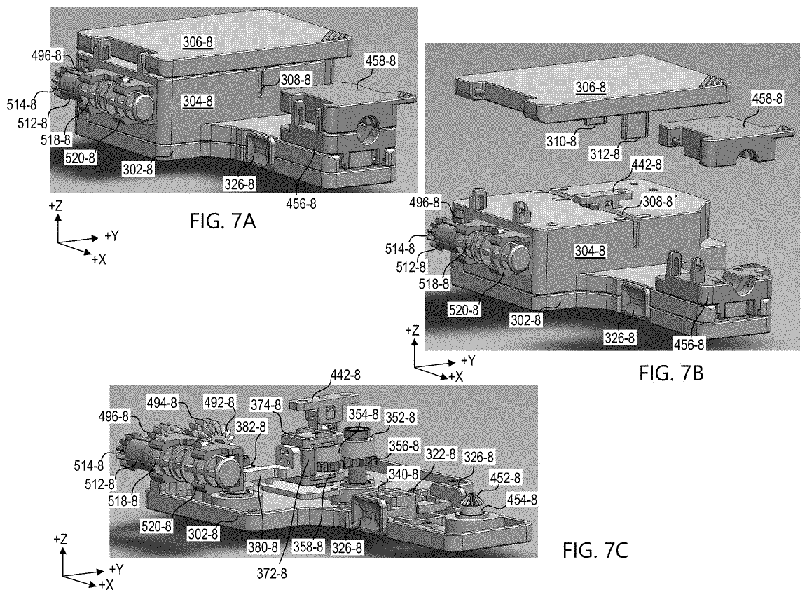

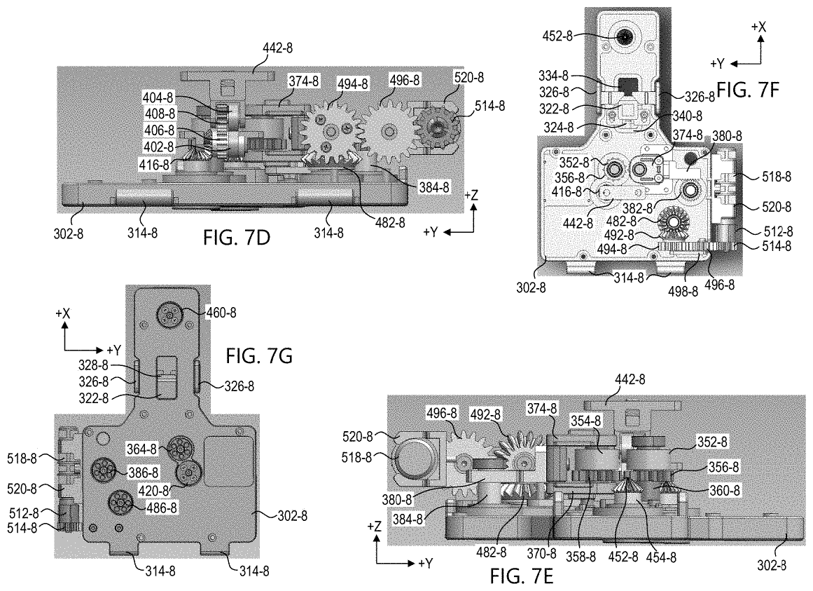

FIG. 7A is a perspective view of a proximal cassette 148 according to some examples. FIGS. 7B and 7C are respective perspective views of the proximal cassette 148 of FIG. 7A partially disassembled according to some examples. FIGS. 7D, 7E, 7F, and 7G are back, front, top, and bottom views, respectively, of the partially disassembled proximal cassette 148 of FIG. 7C. The cassettes shown in FIGS. 7A-7G include some common components. To avoid redundant description, components in the figures are appended with a “−8”, “−4”, or “−2” to indicate in which of the proximal cassette 148, intermediate cassette, or distal cassette 142, respectively, a given component is included. However, description of such components may be without reference to the appended “−8”, “−4”, or “−2”. Various modifications to such components between the different cassettes, including different orientations, may be apparent to a person having ordinary skill, such as to accommodate different components, to accommodate different sized catheters or guidewire, etc.

Each cassette of the proximal cassette 148, intermediate cassettes 144, 146, and distal cassette 142 includes a base 302, a housing 304, and a lid 306. The base 302, housing 304, and lid 306 mechanically support various components and can be any appropriate material, such as molded plastic, with structural integrity to mechanically support those components. The housing 304 is fixedly mechanically attached to the base 302, and the lid 306 is hingedly attached to the housing 304. A channel 308 extends through the housing 304. The channel 308 through the housing 304 extends in the direction that the respective endovascular insertion device advances (e.g., in an X-direction with reference to FIG. 2). The channel 308 is configured to pass an endovascular insertion device therethrough, i.e., the guidewire or a catheter. In the case of, for example, the distal cassette 142, up to three catheters telescoped over on another and a central guideware extend through the channel 308, with only the outer surface of the outermost catheter exposed to the walls of the channel 308. The lid 306 includes a proximal restrictor 310 and a distal restrictor 312 that are configured to, when the lid 306 is closed on the housing 304, project into the channel 308 to restrict vertical movement of the endovascular insertion device therein in operation thereof.

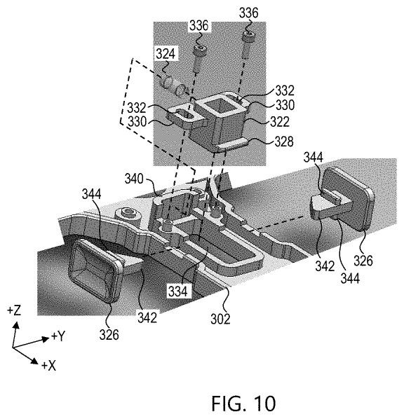

Each cassette includes tabs 314 and a clasp assembly. The tabs 314 project from the base 302 and are configured to engage the hooks 204 when the respective cassette is secured to an appropriate drive unit. FIG. 10 illustrates an exploded perspective view of a clasp assembly according to some examples. The clasp assembly includes a clasp 322, a spring 324, and opposing buttons 326. The clasp 322 is generally a block with a rib 328 and flanges 330. The rib 328 has a lower inclined planar surface (e.g., inversely inclined relative to the inclined planar surface of the respective rib 208) and an upper planar surface. The rib 328 is oriented facing the clasp rib 206 of the appropriate drive unit when secured to the drive unit. Each flange 330 has an elongated opening 332 therethrough. The clasp 322 is disposed at least partially in an opening 334 through the base 302. Respective screws 336 pass through the elongated openings 332 of the flanges 330 of the clasp 322 to secure the clasp 322 disposed at least partially in the opening 334. The elongated openings 332 permit the clasp 322 to be laterally translated. The spring 324 is disposed between a wall 340 of the base 302 and the clasp 322 opposite from the rib 328. The spring 324 is positioned and configured to exert counter forces on the wall 340 and the clasp 322.

Each of the buttons 326 has an angled tab 342 projecting from the respective button 326. Restrictors 344 project from the angled tabs 342. Assembled, the base 302 and housing 304 have walls having openings through which respective angled tabs 342 extend towards the clasp 322. The restrictors 344, in conjunction with these walls of the base 302 and housing 304, restrict movement of the buttons 326.

Assembled and in the absence of any other force, the spring 324 exerting a force on the clasp 322 causes the clasp 322 to be positioned in the opening 334 distally from the wall 340. When the cassette is secured to a drive unit, the tabs 314 are first engaged with the hooks 204, and the clasp assembly is lowered to the clasp rib 206. The respective inclined surfaces of the ribs 208, 328 contact, and as the cassette is lowered, the clasp 322 is displaced more proximally toward the wall 340 to allow the rib 328 to clear the rib 208. Once the rib 328 clears, the spring 324 causes the clasp 322 to be displaced more distally such that the ribs 208, 328 engage each other. This causes the cassette to be secured to the drive unit. To remove the cassette from the drive unit, the buttons 326 are depressed inwardly to the cassette, which causes the angled tabs 342 to displace the clasp 322 more proximally toward the wall 340. This permits the rib 328 to clear the rib 208, which permits the cassette to be removed.

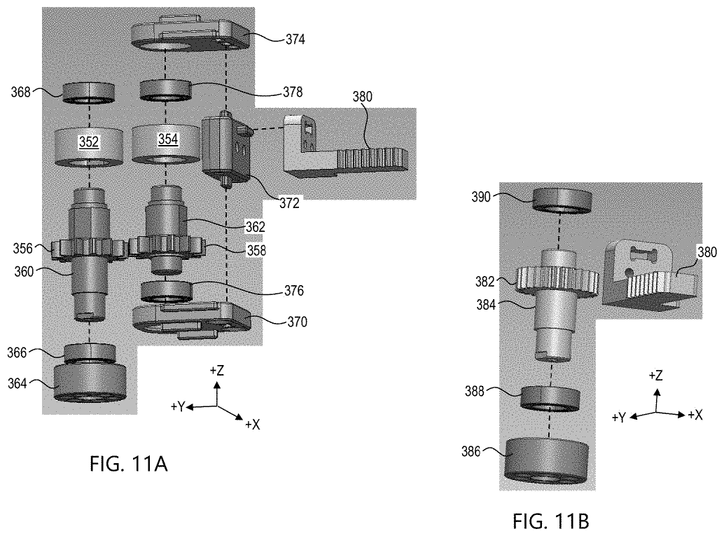

Each cassette includes a pinch and advance assembly. FIGS. 11A and 11B illustrate exploded perspective views of respective portions of a pinch and advance assembly according to some examples. The pinch and advance assembly includes advance rollers 352, 354, advance spur gears 356, 358, and advance shafts 360, 362. The roller surfaces of the advance rollers 352, 354 oppose each other. In operation, the endovascular insertion device is disposed between the roller surfaces of the advance rollers 352, 354. The channel 308 of the housing 304 has respective openings in walls that form the channel 308, which permits the roller surfaces of the advance rollers 352, 354 to contact the endovascular insertion device in the channel 308 and advance the endovascular insertion device.

In the illustrated example, the advance shaft 360 is integral to the advance spur gear 356, and the advance shaft 362 is integral to the advance spur gear 358. In other examples, one or both of the advance shafts 360, 362 can be a separate component from the respective advance spur gear 356, 358. The advance spur gear 356 is disposed on and encircles the advance shaft 360, and the advance spur gear 358 is disposed on and encircles the advance shaft 362. The advance roller 352 is disposed on and encircles the advance shaft 360, and the advance roller 354 is disposed on and encircles the advance shaft 362. Each of the advance shafts 360, 362 can have one or more flat surfaces (e.g., having a D-shaped cross-section normal to an axis of rotation of the advance shafts 360, 362) where the respective advance roller 352, 354 is disposed on the advance shaft 360, 362. Similarly, each of the advance rollers 352, 354 can have an opening having a cross-section that corresponds to the cross-section of the respective advance shaft 360, 362 to help ensure that the advance rollers 352, 354 rotate with the rotation of the respective advance shaft 360, 362.

A female connector 364 is disposed on and mechanically attached to the advance shaft 360. The advance shaft 360 can have one or more flat surfaces (e.g., having a D-shaped cross-section normal to an axis of rotation of the advance shaft 360) where the female connector 364 is disposed on the advance shaft 360. Similarly, the female connector 364 can have an opening having a cross-section that corresponds to the cross-section of the advance shaft 360 to help ensure that the advance shaft 360 rotates with the rotation of the female connector 364. The female connector 364 is exposed and/or extends through the base 302 of the cassette.

A ball bearing 366 mechanically couples the advance shaft 360 to the base 302 of the cassette, and a ball bearing 368 mechanically couples the advance shaft 360 to the housing 304 of the cassette. The ball bearings 366, 368 permit free rotation of the advance shaft 360 while being secured within the cassette.

The pinch and advance assembly includes a pinch support frame that includes, in the illustrated example, a lower support frame 370, an intermediate support frame 372, and an upper support frame 374. The lower support frame 370 is mechanically attached to a lower side of the intermediate support frame 372, and the upper support frame 374 is mechanically attached to an upper side of the intermediate support frame 372. A ball bearing 376 mechanically couples the advance shaft 362 to the lower support frame 370, and a ball bearing 378 mechanically couples the advance shaft 362 to the upper support frame 374. The ball bearings 376, 378 permit free rotation of the advance shaft 362 while being secured between the lower support frame 370 and the upper support frame 374 of the pinch support frame.

A rack 380 is mechanically attached to the pinch support frame (e.g., to the intermediate support frame 372). The rack 380 extends laterally away from the pinch support frame in a direction perpendicular to the axis of rotation of the advance shaft 362. A pinion 382 engages the rack 380. The pinion 382 is disposed on and encircles a pinch shaft 384. In the illustrated example, the pinch shaft 384 is integral to the pinion 382. In other examples, the pinch shaft 384 can be a separate component from the pinion 382. A female connector 386 is disposed on and mechanically attached to the pinch shaft 384. The pinch shaft 384 can have one or more flat surfaces (e.g., having a D-shaped cross-section normal to an axis of rotation of the pinch shaft 384) where the female connector 386 is disposed on the pinch shaft 384. Similarly, the female connector 386 can have an opening having a cross-section that corresponds to the cross-section of the pinch shaft 384 to help ensure that the pinch shaft 384 rotates with the rotation of the female connector 386. The female connector 386 is exposed and/or extends through the base 302 of the cassette.

A ball bearing 388 mechanically couples the pinch shaft 384 to the base 302 of the cassette, and a ball bearing 390 mechanically couples the pinch shaft 384 to the housing 304 of the cassette. The ball bearings 388, 390 permit free rotation of the pinch shaft 384 while being secured within the cassette.

When a cassette is secured to a respective drive unit, the female connector 364 engages the male connector 244a of the advance drive assembly 220a of the drive unit, and the female connector 386 engages the male connector 244b of the pinch drive assembly 220b of the drive unit. In this example configuration, the longitudinal axis of the advance shaft 360 (e.g., around with the advance shaft 360 rotates) is aligned with the transverse axis of the advance drive assembly 220a, and the longitudinal axis of the pinch shaft 384 (e.g., around with the pinch shaft 384 rotates) is aligned with the transverse axis of the pinch drive assembly 220b.

Rotation of the transverse shaft 230b of the pinch drive assembly 220b causes rotation of the pinch shaft 384 (e.g., via the male connector 244b and the female connector 386). Rotation of the pinch shaft 384 causes rotation of the pinion 382, which causes lateral translation of the rack 380 along a direction that the rack 380 extends. Lateral translation of the rack 380 causes lateral translation of the pinch support frame, and thereby, lateral translation of the advance shaft 362 and advance roller 354. Walls and/or surfaces of the housing 304 and/or base 302, and/or other components in the cassette, can restrict the pinch support frame from significant lateral and vertical movement in any direction perpendicular to the direction that the rack 380 extends from the pinch support frame. Additionally, walls, slots, tracks, and/or surfaces of the housing 304 and/or base 302, and/or other components in the cassette, can restrict the amount of lateral movement of the pinch support frame in the direction that the rack 380 extends from the pinch support frame to help prevent over-travel of the pinch support frame.

The configuration of, among other things, the pinch support frame, rack 380, and pinion 382 permit the advance roller 354 and advance shaft 362 to be in at least two positions. In a release position of the advance roller 354 and advance shaft 362, the advance roller 354 is distal from the advance roller 352. In the release position, the endovascular insertion device is released from between the advance rollers 352, 354. No force is exerted on the endovascular insertion device by the advance rollers 352, 354 when the advance roller 354 is at the release position. Further, in the release position, the advance spur gear 358 may be disengaged from the advance spur gear 356. In a pinch position of the advance roller 354 and advance shaft 362, the advance roller 354 is proximate to the advance roller 352. In the pinch position, the endovascular insertion device is pinched, and thereby mechanically coupled and secured, by the advance rollers 352, 354. The advance rollers 352, 354 can exert opposing forces on the endovascular insertion device to pinch the endovascular insertion device. In the pinch position, the advance spur gear 358 engages the advance spur gear 356.

In the pinch position, the pinch and advance assembly can advance the endovascular insertion device. Rotation of the transverse shaft 230a of the advance drive assembly 220a causes rotation of the advance shaft 360 (e.g., via the male connector 244a and the female connector 364). Rotation of the advance shaft 360 causes rotation of the advance spur gear 356, which causes rotation in a counter rotational direction of the advance spur gear 358 and advance shaft 362. The counter rotation of the advance shafts 360, 362 cause counter rotation of the advance rollers 352, 354. Since the advance rollers 352, 354 pinch the endovascular insertion device in this pinch position, the rotation of the advance rollers 352, 354 cause the endovascular insertion device to advance (e.g., to feed the respective catheter into a body or to retrieve the catheter from the body).

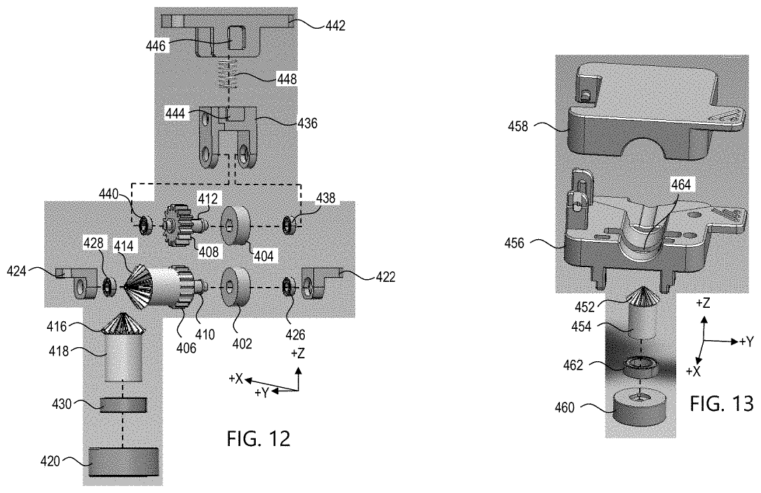

Each cassette includes a follower assembly. FIG. 12 illustrates an exploded perspective view of a follower assembly according to some examples. The follower assembly includes follower rollers 402, 404, follower spur gears 406, 408, follower shafts 410, 412, beveled gears 414, 416, shaft 418, and encoder coupler 420. The roller surfaces of the follower rollers 402, 404 oppose each other. In operation, the endovascular insertion device is disposed between the roller surfaces of the follower rollers 402, 404. The channel 308 of the housing 304 has openings in walls that form the channel 308, which permits the roller surfaces of the follower rollers 402, 404 to contact the endovascular insertion device.

In the illustrated example, the follower shaft 410 is integral to the follower spur gear 406, and the follower shaft 412 is integral to the follower spur gear 408. In other examples, one or both of the follower shafts 410, 412 can be a separate component from the respective follower spur gear 406, 408. The follower spur gear 406 is disposed on and encircles the follower shaft 410, and the follower spur gear 408 is disposed on and encircles the follower shaft 412. The follower roller 402 is disposed on and encircles the follower shaft 410, and the follower roller 404 is disposed on and encircles the follower shaft 412. Each of the follower shafts 410, 412 can have one or more flat surfaces (e.g., having a D-shaped cross-section normal to an axis of rotation of the follower shafts 410, 412) where the respective follower roller 402, 404 is disposed on the follower shaft 410, 412. Similarly, each of the follower rollers 402, 404 can have an opening having a cross-section that corresponds to the cross-section of the respective follower shaft 410, 412 to help ensure that the follower rollers 402, 404 rotate with the rotation of the respective follower shaft 410, 412. The beveled gear 414 is mechanically attached to the follower spur gear 406 and/or the follower shaft 410. As illustrated, the beveled gear 414 is integral with the follower spur gear 406, although in other examples the beveled gear 414 may be a separate component from the follower spur gear 406. Brackets 422, 424 mechanically couple the assembled follower shaft 410, follower roller 402, follower spur gear 406, and beveled gear 414 to the base 302 and/or housing 304 of the cassette. A ball bearing 426 mechanically couples the follower shaft 410 to the bracket 422, and a ball bearing 428 mechanically couples the beveled gear 414 to the bracket 424. The ball bearings 426, 428 permit free rotation of the assembled follower shaft 410, follower roller 402, follower spur gear 406, and beveled gear 414 while being secured within the cassette.

In the illustrated example, the shaft 418 is integral to the beveled gear 416. In other examples, the shaft 418 can be a separate component from the beveled gear 416. The beveled gear 416 is disposed on an end of the shaft 418. The beveled gear 416 is engaged with the beveled gear 414. The encoder coupler 420 is disposed on and mechanically attached to the shaft 418. The shaft 418 can have one or more flat surfaces (e.g., having a D-shaped cross-section normal to an axis of rotation of the shaft 418) where the encoder coupler 420 is disposed on the shaft 418. Similarly, the encoder coupler 420 can have an opening having a cross-section that corresponds to the cross-section of the shaft 418 to help ensure that the shaft 418 rotates with the rotation of the encoder coupler 420. The encoder coupler 420 is exposed and/or extends through the base 302 of the cassette. A ball bearing 430 mechanically couples the shaft 418 to the base 302 of the cassette. The ball bearing 430 permits free rotation of the shaft 418 while being secured within the cassette.

A frame 436 mechanically couples the assembled follower shaft 412, follower roller 404, and follower spur gear 408. Ball bearings 438, 440 mechanically couple the follower shaft 412 to the frame 436. The ball bearings 438, 440 permit free rotation of the assembled follower shaft 412, follower roller 404, and follower spur gear 408 while being secured within the cassette. The frame 436 is mechanically coupled to a bracket 442. The bracket 442 is mechanically attached to a bottom side of the lid 306 of the cassette. The frame 436 is vertically moveable in the bracket 442. The frame 436 includes opposing tabs 444 (one of which is occluded in FIG. 12) projecting laterally in opposite directions from the frame 436, and the bracket 442 has elongated openings 446 through respective lateral sides. Each tab 444 of the frame 436 is inserted into a respective elongated opening 446 of the bracket 442, which mechanically couples the frame 436 to the bracket 442. The elongated openings 446 permit vertical movement of the tabs 444 within the elongated openings 446, which permits vertical movement of the frame 436 with respect to the bracket 442. A spring 448 is disposed vertically between a top side of the frame 436 and an under side of the bracket 442. In the absence of another force, the spring 448 causes the frame 436 to be in a distal position relative to the bracket 442.

When a cassette is secured to a respective drive unit, the encoder coupler 420 engages the encoder coupler 264 of the drive unit. An endovascular insertion device can be placed between the follower rollers 402, 404 by lifting or removing the lid 306 of the cassette and placing the endovascular insertion device in the channel 308 of the cassette. Lifting or removing the lid 306 displaces the follower roller 404, follower shaft 412, follower spur gear 408, frame 436, and bracket 442 to clear the follower roller 404 surface from contact with the follower roller 402 with the channel 308, allowing the endovascular insertion device to be placed in the channel 308 between follower rollers 402, 404. Subsequently replacing or closing the lid 306 causes the follower roller 404, follower shaft 412, follower spur gear 408, frame 436, and bracket 442 to move, causing the surface of the follower roller 404 to move inwardly of the channel 308. The endovascular insertion device is then disposed between the follower rollers 402, 404. The spring 448 causes the follower rollers 402, 404 to exert opposing forces on the endovascular insertion device to restrict vertical movement of the endovascular insertion device. The opposing forces exerted on the endovascular insertion device by the follower rollers 402, 404 are sufficiently large in magnitude to restrict vertical movement of the endovascular insertion device and to cause the follower rollers 402, 404 to rotate with advancement of the endovascular insertion device, and sufficiently small in magnitude to permit rotation of the endovascular insertion device between the follower rollers 402, 404. Typically, when the lid 306 is replaced or closed, the follower spur gear 408 engages the follower spur gear 406.

In operation, as the endovascular insertion device is advanced by the pinch and advance assembly of the cassette, the follower rollers 402, 404 are caused to rotate in counter directions by the advancement of the endovascular insertion device. The rotation of the follower rollers 402, 404 cause the follower shafts 410, 412, and correspondingly, the follower spur gears 406, 408 to rotate. The rotation of the follower rollers 402, 404 and follower shafts 410, 412 can operate cooperatively as a result of the follower spur gear 408 engaging the follower spur gear 406. Rotation of the follower shaft 410 and/or follower spur gear 406 causes the beveled gear 414 to rotate around an axis of rotation around which the follower shaft 410 rotates. Rotation of the beveled gear 414 causes rotation of the beveled gear 416 and shaft 418. The rotation of the beveled gear 416 and shaft 418 is around an axis of rotation that is transverse to the axis of rotation of the beveled gear 414, follower shaft 410, follower spur gear 406, and follower roller 402. The shaft of the encoder 262 is rotated by rotation of the shaft 418 (e.g., via encoder couplers 264, 420). Rotation of the shaft of the encoder 262 can be detected by the encoder 262 and used to extrapolate a length, direction, and/or rate of advancement of the endovascular insertion device by a controller (e.g., a processor). Hence, the follower assembly and encoder 262 can be implemented for feedback control for advancing an endovascular insertion device.

Each of the intermediate cassettes 144, 146 and the distal cassette 142 includes a catheter rotation assembly. FIG. 13 illustrates an exploded perspective view of a catheter rotation assembly according to some examples. The catheter rotation assembly includes a Y-connector housing, a beveled gear 452, and a rotational shaft 454. The Y-connector housing includes a base 456 and a lid 458. The base 456 is mechanically attached to the housing 304 of the cassette. The lid 458 is attached by a hinge to the base 456. The base 456 and lid 458 are configured to secure a Y-connector between the base 456 and lid 458 when the lid 458 is closed on the base 456.

In the illustrated example, the rotational shaft 454 is integral to the beveled gear 452. In other examples, the rotational shaft 454 can be a separate component from the beveled gear 452. The beveled gear 452 is disposed on an end of the rotational shaft 454. A female connector 460 is disposed on and mechanically attached to the rotational shaft 454. The rotational shaft 454 can have one or more flat surfaces (e.g., having a D-shaped cross-section normal to an axis of rotation of the rotational shaft 454) where the female connector 460 is disposed on the rotational shaft 454. Similarly, the female connector 460 can have an opening having a cross-section that corresponds to the cross-section of the rotational shaft 454 to help ensure that the rotational shaft 454 rotates with the rotation of the female connector 460. The female connector 460 is exposed and/or extends through the base 302 of the cassette. A ball bearing 462 mechanically couples the rotational shaft 454 to the base 302 of the cassette. The ball bearing 462 permits free rotation of the rotational shaft 454 while being secured within the cassette.

When the cassette is secured to a respective drive unit, the female connector 460 engages the male connector 244c of the catheter rotational drive assembly 220c of the drive unit. In this example configuration, the longitudinal axis of the rotational shaft 454 (e.g., around which the rotational shaft 454 rotates) is aligned with the transverse axis of the catheter rotational drive assembly 220c.

Rotation of the transverse shaft 230c of the catheter rotational drive assembly 220c causes rotation of the rotational shaft 454 (e.g., via the male connector 244c and the female connector 460). Rotation of the rotational shaft 454 causes rotation of the beveled gear 452. The beveled gear 452, in operation, is engaged (through an opening 464 through the base 456) with another beveled gear mechanically coupled to the catheter that is attached to the Y-connector secured by the Y-connector housing. Rotation of the beveled gear 452 causes rotation of the beveled gear mechanically coupled to the catheter, which causes rotation of the catheter, as will be described in more detail subsequently. Rotation of the beveled gear mechanically coupled to the catheter is around an axis that transverses the axis around which the rotational shaft 454 rotates.

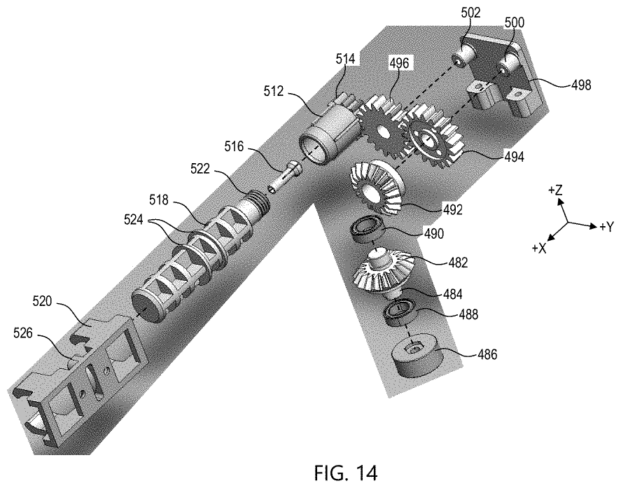

The proximal cassette 148 includes a guidewire rotation assembly. FIG. 14 illustrates an exploded perspective view of a guidewire rotation assembly according to some examples. The guidewire rotation assembly includes a beveled drive gear 482 and a rotational shaft 484. In the illustrated example, the rotational shaft 484 is integral to the beveled drive gear 482. In other examples, the rotational shaft 484 can be a separate component from the beveled drive gear 482. A female connector 486 is disposed on and mechanically attached to the rotational shaft 484. The rotational shaft 484 can have one or more flat surfaces (e.g., having a D-shaped cross-section normal to an axis of rotation of the rotational shaft 484) where the female connector 486 is disposed on the rotational shaft 484. Similarly, the female connector 486 can have an opening having a cross-section that corresponds to the cross-section of the rotational shaft 484 to help ensure that the rotational shaft 484 rotates with the rotation of the female connector 486. The female connector 486 is exposed and/or extends through the base 302 of the cassette. A ball bearing 488 mechanically couples the rotational shaft 484 to the base 302 of the cassette, and a ball bearing 490 mechanically couples the rotational shaft 484 to the housing 304 of the cassette. The ball bearings 488, 490 permit free rotation of the rotational shaft 484 while being secured within the cassette.

The guidewire rotation assembly further includes a driven beveled gear 492, which meshes with the drive bevel gear 482, first and second spur gears 494, 496, and a bracket 498. The bracket 498 is mechanically attached to the base 302 of the proximal cassette 148. The bracket 498 has projections 500, 502. The first spur gear 494 is mechanically coupled to and rotatable around the projection 500, and the second spur gear 496 is mechanically coupled to and rotatable around the projection 502. The first spur gear 494 is engaged with (meshes with) the second spur gear 496. The first beveled gear 492 is mechanically attached to the first spur gear 494. The respective axes of rotation of the first beveled gear 492 and the first spur gear 494 are co-linear.

The guidewire rotation assembly includes a cap 512, a cap spur gear 514, a collet 516, a guidewire connector 518, and a clamping bracket 520. The cap spur gear 514 is mechanically attached at an end of the cap 512. In the illustrated example, the cap spur gear 514 is integral to the cap 512, and in other examples, the cap spur gear 514 and cap 512 can be separate components. The cap 512 includes a threaded female connector (occluded in FIG. 14). The guidewire connector 518 includes a threaded male connector 522. Assembled, the threaded female connector of the cap 512 engages the threaded male connector 522 of the guidewire connector 518. The guidewire connector 518 has a recess with tapered walls interior to the threaded male connector 522. The tapered walls of the guidewire connector 518 generally correspond to angled surfaces of the collet 516. Assembled, the collet 516 is inserted in the recess of the guidewire connector 518, and the threaded female connector of the cap 512 is then engaged with threaded male connector 522 of the guidewire connector 518. As the cap 512 is rotated on the guidewire connector 518 by the threads engaging, the collet 516 is compressed. A guidewire can be threaded through the collet 516 and an opening through the cap 512 such that the collet 516 clamps and secures the guidewire by the collet 516 being compressed.

The clamping bracket 520 is mechanically attached to the housing 304 of the proximal cassette 148. The clamping bracket 520 is configured to hold the guidewire connector 518 while allowing the guidewire connector 518 to rotate. The guidewire connector 518 has ribs 524 around the exterior of the guidewire connector 518. The clamping bracket 520 has a restrictor 526. When the clamping bracket 520 holds the guidewire connector 518, the restrictor 526 is disposed laterally between the ribs 524 of the guidewire connector 518, which can restrict significant lateral movement of the guidewire connector 518. The clamping bracket 520 permits rotation of the guidewire connector 518 around a longitudinal axis of the guidewire connector 518. When the clamping bracket 520 holds the guidewire connector 518 with the cap 512 being disposed on the guidewire connector 518, the spur gear 514 engages the spur gear 496.

When the proximal cassette 148 is secured to the proximal drive unit 138, the female connector 486 engages the male connector 244d of the guidewire rotational drive assembly 220d of the proximal drive unit 138. In this example configuration, the longitudinal axis of the rotational shaft 484 (e.g., around which the rotational shaft 484 rotates) is aligned with the transverse axis of the guidewire rotational drive assembly 220d.

Rotation of the transverse shaft 230d of the guidewire rotational drive assembly 220d causes rotation of the rotational shaft 484 (e.g., via the male connector 244d and the female connector 486). Rotation of the rotational shaft 484 causes rotation of the drive beveled gear 482, which causes rotation of the driven beveled gear 492 around an axis that is transverse to the transverse axis of the guidewire rotational drive assembly 220d. Rotation of the driven beveled gear 492 causes rotation of the first spur gear 494 in a same direction. Rotation of the first spur gear 494 causes rotation of the second spur gear 496 in a counter direction, in other words, in an opposite direction. Rotation of the second spur gear 496 causes rotation of the cap spur gear 514 in a counter direction, which, when the collet 516 is clamped onto a guidewire extending therethrough, causes the guidewire to rotate.

FIGS. 15, 16, and 17 illustrate configurations that include a catheter and a Y-connector. In FIG. 15, a Y-connector 602 includes a female connector of a Luer lock. The female connector of a Luer lock has a connector beveled gear 604 integral with the outer sheath of the female connector. A catheter 606 has tabs and a male connector of a Luer lock at a proximal end of the catheter 606. The male connector of the catheter 606, in operation, is engaged with the female connector of the Y-connector 602. Rotation of the connector beveled gear 604 on the sheath of the female connector causes rotation of the catheter 606.

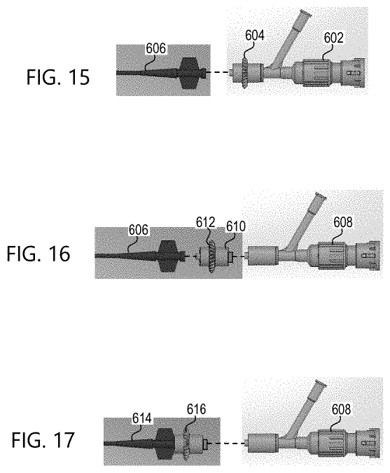

In FIG. 16, a Y-connector 608 includes a female connector of a Luer lock. A catheter 606 has tabs and a male connector of a Luer lock at a proximal end of the catheter 606. An intermediate connector 610 has a female connector and a male connector of a Luer lock. The intermediate connector 610 has an intermediate connector beveled gear 612 integral with the outer sheath of the intermediate connector 610. The male connector of the catheter 606, in operation, is engaged with the female connector of the intermediate connector 610, and the male connector of the intermediate connector 610 is engaged with the female connector of the Y-connector 608. Rotation of the intermediate connector beveled gear 612 on the intermediate connector 610 causes rotation of the catheter 606.

In FIG. 17, a Y-connector 608 includes a female connector of a Luer lock. A catheter 614 has tabs and a male connector of a Luer lock at a proximal end of the catheter 614. The male connector of the catheter 614 has a Luer beveled gear 616 integral with the outer sheath of the male connector. The male connector of the catheter 614, in operation, is engaged with the female connector of the Y-connector 608. Rotation of the Luer beveled gear 616 on the sheath of the male connector causes rotation of the catheter 614.

Referring back to FIG. 13, the Y-connector housing (including the base 456 and lid 458) can be configured to accommodate and secure a Y-connector, including the Y-connectors 602, 608 of FIGS. 15 through 17. The Y-connector housing is configured such that the beveled gear 604, 612, 616 mechanically coupled to the Y-connector 602, 608 and/or catheter 606, 614 engages the Y-connector beveled gear 452 of the catheter rotation assembly through the opening 464 when the Y-connector housing secures the Y-connector 602, 608. Hence, rotation of the Y-connector beveled gear 452 (by rotation of the rotational shaft 454) causes the beveled gear 604, 612, 616 to rotate around an axis transverse to the axis of rotation of the rotational shaft 454, which further causes the catheter 606, 614 to rotate.

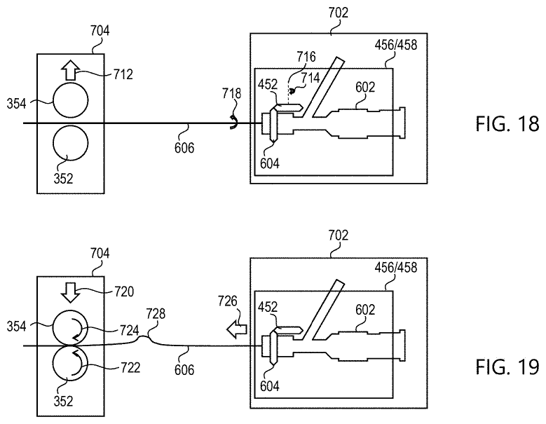

FIGS. 18 and 19 are schematic illustrations depicting rotation and advancement, respectively, of a catheter according to some examples. FIGS. 18 and 19 show a first cassette 702 and a second cassette 704. The first cassette 702 is more proximally positioned, and the second cassette 704 is more distally positioned. The first cassette 702 and the second cassette 704 can be the distal cassette 142 and the first intermediate cassette 144, respectively. The first cassette 702 and the second cassette 704 can be the first intermediate cassette 144 and the second intermediate cassette 146, respectively. The first cassette 702 and the second cassette 704 can be the second intermediate cassette 146 and the proximal cassette 148, respectively.

The first cassette 702 is shown including a Y-connector housing (including the base 456 and lid 458) that secures the Y-connector 602 with the beveled gear 604. The catheter 606 is engaged with the Y-connector 602. The Y-connector housing can secure any Y-connector and beveled gear configuration, and any catheter can be implemented in other examples. The beveled gear 604 is engaged with the beveled gear 452 of the catheter rotation assembly of the first cassette 702. The catheter 606 extends through the channel 308 of the second cassette 704 (e.g., between the advance rollers 352, 354).

In operation, the pinch and advance assembly of the second cassette 704 can secure the catheter 606 in the channel 308 of the second cassette 704 for no movement or for advancement in a lateral direction. If the catheter 606 is to be rotated, the pinch and advance assembly of the second cassette 704 releases the catheter 606 for rotation. Regardless of the movement of the catheter 606 (e.g., rotation, advancement, and no movement), the catheter rotation assembly of the first cassette 702, which is configured to rotate the catheter 606, can remain mechanically coupled to the catheter 606 (e.g., by the beveled gear 452 engaging the beveled gear 604 that is mechanically coupled to the catheter 606).

First, it is assumed that the first cassette 702 and second cassette 704 are in respective positions where the catheter 606 is not being moved, such that the pinch and advance assembly of the second cassette 704 causes the catheter 606 to be secured between the advance rollers 352, 354 of the second cassette 704. To rotate the catheter 606, the pinch and advance assembly of the second cassette 704 releases the catheter 606. The advance roller 354 of the second cassette 704 is laterally translated such that the opposing advance rollers 352, 354 do not apply opposing forces (e.g., pinch) to the catheter 606. With reference to FIGS. 11A and 11B, the pinion 382 is rotated by the pinch drive assembly 220b (e.g., via male connector 244b and female connector 386), and rotation of the pinion 382 causes the rack 380 to be translated such that the pinch support frame (including the lower support frame 370, intermediate support frame 372, and upper support frame 374) and advance roller 354 are translated in a direction away from the advance roller 352 (e.g., in a —Y direction). With reference to FIG. 18, the advance roller 354 is translated in direction 712 away from the advance roller 352 to a release position.

With the catheter 606 released by the pinch and advance assembly, the catheter rotation assembly of the first cassette 702 can rotate the catheter 606. To rotate the catheter 606, the beveled gear 452 of the first cassette 702 is rotated 714 around an axis 716. The rotation 714 of the beveled gear 452 of the first cassette 702 causes the beveled gear 604 to rotate around a transverse axis, which causes the catheter 606 to rotate 718. With reference to FIG. 13, the beveled gear 452 is rotated by the catheter rotational drive assembly 220c (e.g., via male connector 244c and female connector 460). The axis 716 corresponds to the longitudinal axis of the rotational shaft 454 around which the rotational shaft 454 and beveled gear 452 rotate.

To advance the catheter 606, with reference to FIG. 19, the pinch and advance assembly of the second cassette 704 pinches the catheter 606. The advance roller 354 of the second cassette 704 is laterally translated such that the opposing advance rollers 352, 354 apply opposing forces (e.g., pinch) to the catheter 606. With reference to FIGS. 11A and 11B, the pinion 382 is rotated by the pinch drive assembly 220b (e.g., via male connector 244b and female connector 386), and rotation of the pinion 382 causes the rack 380 to be translated such that the pinch support frame (including the lower support frame 370, intermediate support frame 372, and upper support frame 374) and advance roller 354 are translated in a direction towards the advance roller 352 (e.g., in a +Y direction). With reference to FIG. 19, the advance roller 354 is translated in direction 720 towards the advance roller 352 to a pinch position.

With the catheter 606 pinched by the pinch and advance assembly, the pinch and advance assembly of the second cassette 704 can advance (e.g., feed into a body or retrieve from the body) the catheter 606. To advance the catheter 606, the advance shaft 360 of the second cassette 704 is rotated by the advance drive assembly 220a (e.g., via male connector 244a and female connector 364), causing the advance roller 352 to rotate 722. Further, the rotation of the advance shaft 360 of the second cassette 704 causes the advance spur gear 356 to likewise rotate. In the pinch position, the advance spur gear 356 engages the advance spur gear 358. Hence, rotation of the advance spur gear 356 causes counter-rotation of the advance spur gear 358, which causes counter-rotation 724 of the advance roller 354. The rotations 722, 724 of the advance rollers 352, 354 shown in FIG. 19 can cause the catheter 606 to be fed into a body. Rotations of the advance rollers 352, 354 opposite to the respective illustrated rotations 722, 724 can cause the catheter to be retrieved from a body.

When the pinch and advance assembly of the second cassette 704 advances the catheter 606, the first cassette 702 follows the advancement of the catheter 606. As shown in FIG. 19, the first cassette 702 follows in lateral translation direction 726 (e.g., when the catheter 606 is fed into a body). The first cassette 702 can follow in a lateral translation direction opposite from the direction 726 (e.g., when the catheter 606 is retrieved from a body). The following of the first cassette 702 can be by an independent translation assembly, such as described subsequently. The following by the first cassette 702 can cause little or no tension to be in the catheter 606 between the advance rollers 352, 354 of the second cassette 704 pinching the catheter 606 and the Y-connector 602 secured by the Y-connector housing of the first cassette 702. Such absence of tension is illustrated in FIG. 19 by slack 728 being present in the catheter 606.

As illustrated by FIGS. 18 and 19, the catheter rotation assembly of the first cassette 702 can remain engaged with or be mechanically coupled to the connector beveled gear 604 (e.g., by the beveled gear 452 engaging the connector beveled gear 604) regardless of the movement of the catheter 606. Hence, the catheter rotation assembly can remain mechanically coupled to the catheter 606 regardless of the movement of the catheter 606 in operation. In FIG. 19, the catheter rotation assembly is not operating to rotate the catheter 606, and the beveled gear 452 remains engaged with the beveled gear 604 while the catheter 606 is advanced. Also, as illustrated by FIGS. 18 and 19, the pinch and advance assembly of the second cassette 704 releases or becomes mechanically de-coupled from the catheter 606 to permit rotation of the catheter 606 through the channel 308 of the second cassette 704.

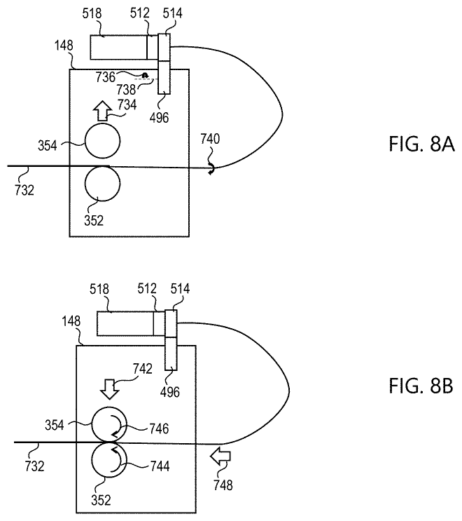

FIGS. 8A and 8B are schematic illustrations depicting rotation and advancement, respectively, of a guidewire 732 according to some examples. FIGS. 8A and 8B show the proximal cassette 148. The proximal cassette 148 is shown including a guidewire connector 518 and cap 512. The guidewire 732 is secured by the guidewire connector 518, cap 512, and collet 516 (not shown), as described previously. The cap spur gear 514 on the cap 512 is engaged with the spur gear 496. The guidewire 732 extends from the guidewire connector 518 and cap 512 through the channel 308 of the proximal cassette 148 (e.g., between the advance rollers 352, 354). The length of the guidewire 732 that extends from the cap 512 to the housing 304 (e.g., to the channel 308 through the housing 304) may be referred to a loop-back of the guidewire 732.

In operation, the pinch and advance assembly of the proximal cassette 148 can secure the guidewire 732 in the channel 308 of the proximal cassette 148 for no movement or for advancement in a lateral direction. If the guidewire 732 is to be rotated, the pinch and advance assembly of the proximal cassette 148 releases the guidewire 732 for rotation. Regardless of the movement of the guidewire 732 (e.g., rotation, advancement, and no movement), the guidewire rotation assembly of the proximal cassette 148 can remain mechanically coupled to the guidewire 732 (e.g., by the collet 516, guidewire connector 518, and cap 512).

First, it is assumed that the proximal cassette 148 is in a position where the guidewire 732 is not being moved, such that the pinch and advance assembly of the proximal cassette 148 causes the guidewire 732 to be secured between the advance rollers 352, 354 of the proximal cassette 148. To rotate the guidewire 732, the pinch and advance assembly releases the guidewire 732. The advance roller 354 of the proximal cassette 148 is laterally translated in a direction 734 to a release position such that the opposing advance rollers 352, 354 do not apply opposing forces (e.g., pinch) to the guidewire 732, like described above with respect to FIG. 18.

With the guidewire 732 released by the pinch and advance assembly of the proximal cassette 148, the catheter rotation assembly of the proximal cassette 148 can rotate the guidewire 732. To rotate the guidewire 732, the spur gear 496 of the proximal cassette 148 is rotated 736 around an axis 738. The rotation 736 of the spur gear 496 causes the cap spur gear 514, and hence, the cap 512, guidewire connector 518, and collet 516, to rotate in a direction opposite the rotation 736, which causes the guidewire 732 to rotate 740. With reference to FIG. 14, the spur gear 496 is rotated by the guidewire rotational drive assembly 220d (e.g., via male connector 244d and female connector 486).

To advance the guidewire 732, with reference to FIG. 21, the pinch and advance assembly of the proximal cassette 148 pinches the guidewire 732. The advance roller 354 is laterally translated such that the opposing advance rollers 352, 354 apply opposing forces (e.g., pinch) to the guidewire 732, like described with reference to FIG. 19. The advance roller 354 is translated in direction 742 towards the advance roller 352 to a pinch position.

With the guidewire 732 pinched by the pinch and advance assembly, the pinch and advance assembly can advance (e.g., feed into a body or retrieve from the body) the guidewire 732. To advance the guidewire 732, the advance shaft 360 of the proximal cassette 148 is rotated by the advance drive assembly 220a (e.g., via male connector 244a and female connector 364), causing the advance roller 352 to rotate 744. Further, the rotation of the advance shaft 360 causes the advance spur gear 356 to likewise rotate. In the pinch position, the advance spur gear 356 engages the advance spur gear 358. Hence, rotation of the advance spur gear 356 causes counter-rotation of the advance spur gear 358, which causes counter-rotation 746 of the advance roller 354. The rotations 744, 746 of the advance rollers 352, 354 shown in FIG. 21 can cause the guidewire 732 to be fed into a body. Rotations of the advance rollers 352, 354 opposite to the respective illustrated rotations 744, 746 can cause the catheter to be retrieved from a body.

When the pinch and advance assembly of the proximal cassette 148 advances the guidewire 732, the loop-back of the guidewire 732 can change. As shown in FIG. 21, the length of the loop-back can be decreased as the guidewire 732 is advanced in a lateral translation direction 748 (e.g., when the guidewire 732 is fed into a body). Similarly, the length of the loop-back can be increased as the guidewire 732 is advanced in a lateral translation direction opposite from the direction 748 (e.g., when the guidewire 732 is retrieved from a body).

As illustrated by FIGS. 8A and 8B, the guidewire rotation assembly of the proximal cassette 148 can remain engaged with or mechanically coupled to the guidewire 732 (e.g., by the guidewire connector 518, collet 516, and cap 512 securing the guidewire 732) regardless of the movement of the guidewire 732. Hence, the guidewire rotation assembly can remain mechanically coupled to the guidewire 732 regardless of the movement of the guidewire 732 in operation. In FIG. 21, the catheter rotation assembly is not operating to rotate the guidewire 732, and the guidewire connector 518, collet 516, and cap 512 remain securing the guidewire 732, with the spur gears 514, 496 remaining engaged, while the guidewire 732 is advanced. Also as illustrated by FIGS. 20 and 21, the pinch and advance assembly of the proximal cassette 148 releases or becomes mechanically de-coupled from the guidewire 732 to permit rotation of the guidewire 732 through the channel 308 of the proximal cassette 148.

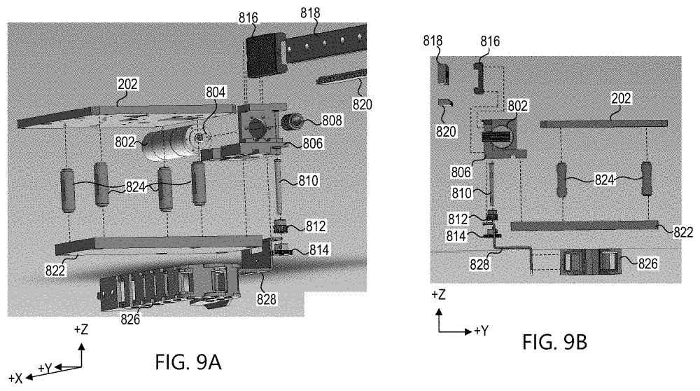

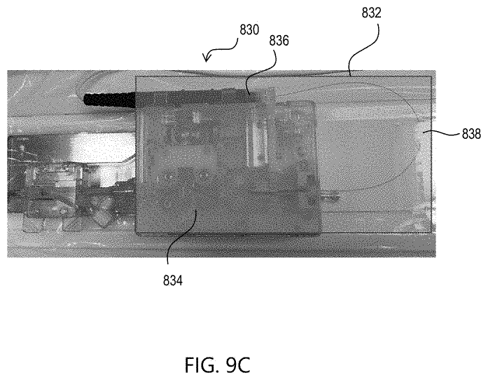

FIGS. 9A to 9C illustrate exploded perspective views of a translation assembly mechanically coupled to a track according to some examples. Each of the first intermediate drive unit 134, second intermediate drive unit 136, and proximal drive unit 138 includes a respective translation assembly mechanically coupled thereto. The translation assemblies permit the respective drive units 134-138 to be translated along the track (e.g., in an X-direction). Different translation assemblies and/or modifications to the illustrated translation assembly may be implemented for the drive units. Various types of shafts and gears are described below with respect to the translation assembly; however, other types of shafts and gears can be implemented to achieve a different configuration of a translation assembly.

The translation assembly includes a rotational actuator 802. The rotational actuator 802 includes a drive shaft 804 (e.g., a shaft having a D-shaped cross-section normal to an axis of rotation of the shaft). The rotational actuator 802 is configured to rotate the drive shaft 804. In some examples, the rotational actuator 802 is a motor, such as an electric motor. The rotational actuator 802 is mechanically attached and mounted on a bracket 806. A worm gear 808 is mechanically attached to the drive shaft 804.

The translation assembly further includes a transverse shaft 810 (e.g., a shaft having a D-shaped cross-section normal to an axis of rotation of the shaft). A gear 812 (e.g., a spur gear with outer faces that are concave) and a pinion 814 are each mechanically attached to and encircle the transverse shaft 810. The worm gear 808 engages the gear 812. The rotational actuator 802 is configured to rotate the drive shaft 804, which causes the worm gear 808 to rotate around a drive axis. Rotation of the worm gear 808 causes rotation of the gear 812 around a transverse axis that is transverse to the drive axis. Rotation of the gear 812 causes the transverse shaft 810 to rotate around the transverse axis, which further causes the pinion 814 to rotate around the transverse axis. The translation assembly also includes a slider 816. The slider 816 generally has a C-shaped cross section, as shown in FIG. 22B. The slider 816 is mechanically attached to the bracket 806.

The track includes a guide 818 and a rack 820. Although not illustrated in FIGS. 9A and 9B, the guide 818 and rack 820 are mechanically coupled to and supported by the frame 112. The guide 818 has a groove on a top side and a groove on a bottom side. The slider 816 engages the grooves of the guide 818 to mechanically support the translation assembly and to permit translation of the translation assembly along the guide 818. The pinion 814 engages the rack 820. Rotation of the pinion 814, by engaging with the rack 820, causes translation of the translation assembly.

The translation assembly is mechanically coupled to the respective drive unit. In FIGS. 9A and 9B, a mounting plate 822 is mechanically attached to the bracket 806. The mounting plate 822 is mechanically attached to spacers 824, which are mechanically attached to the support plate 202 of the respective drive unit.

The translation assembly can also include a linkage conduit 826. In the illustrated example, an end of the linkage conduit 826 is mechanically attached to a bracket 828, which is mechanically attached to the bracket 806. Another end of the linkage conduit 826 is mechanically coupled to the frame 112 (not shown). The linkage conduit 826 can carry wires and cables that transmit power and/or control signals to various electrical components within the translation assembly and drive unit. The end of the linkage conduit 826 mechanically coupled to the frame 112 can remain in a fixed position, while the end of the linkage conduit 826 mechanically attached to the bracket 828 can be moveable with the translation of the translation assembly. The linkage conduit 826 can reduce kinking or tangling of wires or cables carried by the linkage conduit 826.

FIG. 9C schematically shows a view of the proximal cassette having the translation assembly of FIGS. 9A and 9B incorporated within the housing thereof, according to some examples. The proximal cassette 830 herein comprises a housing 832. The pinch and advance assembly 834 as shown in FIG. 11A, the guidewire rotation assembly 834 as shown in FIG. 14 and the guidewire 838 are entirely received within the housing 832.

Although various examples have been described in detail, it should be understood that various changes, substitutions and alterations can be made therein without departing from the scope defined by the appended claims.

What is claimed is:

1. A guidewire controller cassette for moving a guidewire having a proximal end, the guidewire controller comprising:
a housing having an interior space;
a translational module received within the interior space and having an entry side, an opposed exit side and a lateral wall being disposed therebetween, the translational module comprising a first guidewire path extending between the entry side and the exit side and configured to move the guidewire translationally along the first guidewire path; and
a rotational module received within the interior space and mounted at the lateral wall, the rotational module comprising an opening for receiving the proximal end of the guidewire, a rotating axis allowing the proximal end of the guidewire being rotated thereabout, and a second guidewire path which is a loop path extending out from the opening to the entry side.

2. The guidewire controller cassette of claim 1, wherein the translational module comprises at least a pair of rollers, each roller being disposed to face one another along the first guidewire path, and each roller having an outer circumferential surface, at least a portion of which grips the sheath extending between the pair of rollers.

3. The guidewire controller cassette of claim 1, wherein the rotational module further comprises a guidewire connector disposed along the rotating axis and having a collet received within the recess and having an opening for receiving the proximal end of the guidewire.

4. The guidewire controller cassette of claim 3, wherein the rotational module further comprises a first gear assembly disposed at a proximal end of the guidewire controller, a second gear assembly engaged to the first gear assembly along a perpendicular direction of the rotational axis.

5. The guidewire controller cassette of claim 4, wherein the second gear assembly has a rotational shaft extending along the perpendicular direction of the rotational axis.

6. A method for moving a guidewire, comprising:
providing the guidewire controller comprising: a cartridge having an interior space;
a translational module received within the interior space and having an entry side, an opposed exit side and a lateral wall being disposed therebetween, the translational module comprising a first guidewire path extending between the entry side and the exit side and configured to move the guidewire translationally along the first guidewire path; and
a rotational module received within the interior space and mounted at the lateral wall, the rotational module comprising an opening for receiving the proximal end of the guidewire and a longitudinal axis allowing the proximal end of the guidewire being rotated thereabout, and a second guidewire path which is a loop path extending out from the opening to the entry side;
engaging the guidewire to the rotational module and the translational module along the first guidewire path and the second guidewire path; and
moving the guidewire translationally or rotationally along the first guidewire path and the second guidewire path in response to a control signal of the guidewire controller.

7. The method of claim 6, wherein the translational module comprises at least a pair of rollers, each roller being disposed to face one another along the first guidewire path, and each roller having an outer circumferential surface, at least a portion of which grips the sheath extending between the pair of rollers.

8. The method of claim 6, wherein the guidewire is engaged to form a loop along the second guidewire path.
